(19) United States
(12) Patent Application Publication
(10) Pub. No.: US 2025/0057196 A1
(43) Pub. Date: Feb. 20, 2025

(54) CELL PROLIFERATION MEDIUM FOR PRODUCING CULTURED MEAT

(71) Applicant: NH Foods Ltd., Osaka-shi, Osaka (JP)

(72) Inventors: Makoto Segawa, Tsukuba-shi, Ibaraki (JP); Yoshikazu Furusawa, Tsukuba-shi, Ibaraki (JP); Yasutaka Nishiyama, Tsukuba-shi, Ibaraki (JP)

(21) Appl. No.: 18/724,945

(22) PCT Filed: Dec. 28, 2022
(86) PCT No.: PCT/JP2022/048674

§ 371 Date: Jun. 27, 2024

(30) Foreign Application Priority Data
Dec. 28, 2021 (JP) 2021-215213

(51) Int. Cl. A23L 13/00; C12N 5/077

(52) U.S. Cl. CPC A23L 13/00; C12N 5/0653; C12N 5/0656; C12N 5/0658; C12N 2500/76; C12N 2500/84

(57) ABSTRACT

The purpose of the present invention is to provide a medium, in which cells to be used for producing a cultured meat can be proliferated, by adding a food material as an additive. The present inventors cultured cells with the use of food components as additives and, consequently, found that when whey was added as a cell proliferation promoting agent, fibroblasts, adipocytes and myoblasts exhibited high proliferation ability. Thus, the present invention, which pertains to a cell proliferation medium comprising a basic medium and whey as a cell proliferation promoting agent, has been completed.

### TECHNICAL FIELD

The present invention relates to the technical field of producing cultured meat. More specifically, the invention relates to a medium for proliferation of cells to be used for producing cultured meat, a method for producing the medium, a method for preparing cells for producing cultured meat, and a cell proliferation promoting agent for producing cultured meat.

### BACKGROUND ART

Meat has traditionally been produced by raising livestock. Raising of livestock, however, requires large amounts of grains and water, as well as large breeding farms. In recent years, awareness of problems such as climate change and food shortages has led to demand for more sustainable meat production with reduced environmental impact and higher productivity. In view of this situation, the production of cultured meat is attracting attention, as a new method of meat production.

Plant-derived meat substitutes are known, but they still fail to provide the mouthfeel and flavor of meat. Cultured meat obtained by culturing of animal cells can provide a mouthfeel and flavor similar to that of meat, with the added advantage of lower risk of contamination by bacteria and viruses than actual meat. Production of cultured meat has recently become technically feasible. However, the cell culture media currently used for producing cultured meat are for basic research and drug application, and are difficult to use for food production due to issues of cost and safety. For cell culture media used for basic research and drug applications, it is common to add fetal bovine serum (FBS) as an added component to basal medium containing amino acids, vitamins, inorganic salts and carbon sources such as glucose (NPL 1: Mol Ther. 2004 Mar; 9(3):475-82). Because FBS is fetus-harvested serum it is difficult to obtain in mass quantities, while it is also associated with issues of basic cost, transport cost, infectious disease risk and animal welfare. To overcome these issues, chemically defined media have been developed, which contain essential components for cell proliferation (NPL 2: The Canadian Journal of Chem Engineering Vol. 94, (10) October 2016 1855-1862). Such chemically defined media, however, use recombinant proteins, hormone agents and serum-derived components, which raises concerns about safety.

The use of cell culture media for cultured meat production has been attempted using various approaches. These include culture media using factors produced by organ cells (PTL 1: Japanese Patent Publication No. 6111510), culture media using algae products (NPL 3: Scientific Reports. Jan 31; 7:41594), culture media using food residue hydrolysates (NPL 4: Food Funct., 2020, 11, 2477-2488), and culture media using other food material components (PTL 2: International Patent Publication No. WO2021/148955).

### CITATION LIST

### Patent Literature

[PTL 1] Japanese Patent Publication No. 6111510
[PTL 2] International Patent Publication No. WO2021/148955

### Non Patent Literature

[NPL 1] Mol Ther. 2004 March; 9(3):475-82
[NPL 2] The Canadian Journal of Chem Engineering Vol. 94, (10) October 2016 1855-1862
[NPL 3] Scientific Reports. Jan 31; 7:41594
[NPL 4] Food Funct., 2020, 11, 2477-2488

### SUMMARY OF INVENTION

### Technical Problem

It is an object of the present invention to provide a culture medium allowing large-scale culturing of cells to be used for production of cultured meat, by addition of food material components as cell proliferation promoting agents.

### Solution to Problem

The present inventors have ardently studied culture media usable for production of cultured meat, and have completed this invention upon finding that adding whey as a cell proliferation promoting agent to medium allows high proliferative activity to be achieved for cells serving as the starting material for cultured meat. The present invention relates to the following.
[1]A cell proliferation medium containing basal medium and whey as a cell proliferation promoting agent.
[2] The medium according to [1] above, wherein the proliferated cells are cells to be used for producing cultured meat.
[3] The medium according to [1] or [2] above, wherein the medium does not contain animal-derived serum.
[4] The medium according to [3] above, wherein the animal-derived serum is fetal bovine serum (FBS).
[5] The medium according to any one of [1] to [4] above, wherein the cells comprise at least one type of cell selected from the group consisting of fibroblasts, adipose tissue-derived cells and muscle tissue-derived cells.
[6] The medium according to [5] above, wherein the adipose tissue-derived cells are at least one type of cell selected from the group consisting of adipose stem cells, multilocular adipocytes and unilocular adipocytes.
[7] The medium according to [5] above, wherein the muscle tissue-derived cells are at least one type of cell selected from the group consisting of myoblasts and muscle satellite cells.
[8] The medium according to any one of [1] to [7] above, wherein the cells are bovine cells.
[9] The medium according to any one of [1] to [8] above, which further comprises a food material component.
[10] The medium according to [9] above, wherein the food material component is selected from among egg white, soybean, fish powder and wheat flour.
[11]A method for preparing cells for producing cultured meat, comprising a step of culturing cells in medium containing basal medium and whey as a cell proliferation promoting agent.
[12] The method according to [11] above, wherein the medium does not contain animal-derived serum.
[13] The method according to [12] above, wherein the animal-derived serum is fetal bovine serum (FBS).
[14] The method according to any one of [11] to [13] above, wherein the medium further comprises a food material component.
[15] The method according to [14] above, wherein the food material component is selected from among egg white, soybean, fish powder and wheat.
[16] The method according to any one of [11] to [15] above, wherein the cells are bovine cells.
[17] The method according to any one of [11] to [16] above, wherein the cells comprise at least one type of cell selected from the group consisting of fibroblasts, adipose tissue-derived cells and muscle tissue-derived cells.
[18] The method according to [17] above, wherein the adipose tissue-derived cells are at least one type of cell selected from the group consisting of adipose stem cells, multilocular adipocytes and unilocular adipocytes.
[19] The method according to [17] above, wherein the muscle tissue-derived cells are at least one type of cell selected from the group consisting of myoblasts and muscle satellite cells.
[20] The method according to [19] above, wherein the cells are myoblasts or muscle satellite cells, and wherein the method further comprises a differentiation-inducing step from myoblasts or muscle satellite cells to myotube cells.
[21]A method for producing cultured meat, comprising a step of accumulating cells prepared by the method according to any one of [11] to [20] above.
[22] The method according to [21] above, wherein the prepared cells are accumulated together with one or more substances selected from the group consisting of other cells, blood, tissue and extracellular matrix.
[23] The method according to [22] above, wherein the other cells are cultured cells or cells acquired from an animal.
[24] The method according to any one of [21] to [23] above, comprising further culturing after accumulation.
[25]A cell proliferation promoting agent for producing cultured meat, comprising whey.
[26] The cell proliferation promoting agent according to [25] above, which is added to animal-derived serum-free medium.
[27] The cell proliferation promoting agent according to [25] or [26], which promotes proliferation of at least one type of cell selected from the group consisting of fibroblasts, adipose tissue-derived cells and muscle tissue-derived cells.
[28] The cell proliferation promoting agent according to [27] above, wherein the adipose tissue-derived cells are at least one type of cell selected from the group consisting of adipose stem cells, multilocular adipocytes and unilocular adipocytes.
[29] The cell proliferation promoting agent according to [27] above, wherein the muscle tissue-derived cells are at least one type of cell selected from the group consisting of myoblasts and muscle satellite cells.
[30] The cell proliferation promoting agent according to any one of [25] to [29] above, wherein the cells are bovine cells.
[31]A method for producing medium, comprising:
a step of mixing basal medium with whey as a cell proliferation promoting agent to acquire a cell proliferation medium; and a step of heat sterilizing the medium.
[32] The method according to [31] above, wherein the step of heat sterilizing is carried out by boiling treatment.
[33] The method according to [31] above, wherein the step of heat sterilizing is carried out using a heat plate exchanger or a steam sterilizer.

### Advantageous Effects of Invention

By adding whey to basal medium it is possible to provide a cell culture medium that enhances the cell proliferation activity of cells.

### BRIEF DESCRIPTION OF DRAWINGS

FIG. 1 shows the proliferation rates for culturing of primary myoblasts and fibroblasts from 7 cows, when cultured in serum-free medium and in 10% FBS-added medium.

FIG. 2 shows the results of screening for food material components that enhance cell proliferation, when culturing bovine myoblasts in serum-free medium.

FIG. 3 shows the results of screening for food material components that enhance cell proliferation, when culturing bovine adipocytes in serum-free medium.

FIG. 4 shows the results of screening for food material components that enhance cell proliferation, when culturing bovine fibroblasts in serum-free medium. The results shown are for (A) Holstein-derived fibroblasts, (B) first-generation hybrid (F1) bovine fibroblasts and (C) Japanese Black breed-derived fibroblasts.

FIG. 5 is a graph showing the concentration-dependent proliferation promoting effect of using whey as a cell growth proliferation promoting agent for culturing of bovine myoblasts in serum-free medium.

FIG. 6 shows the results of screening for food material components that enhance cell proliferation, when culturing bovine myoblasts in serum-free medium in combination with whey.

FIG. 7 shows the results of screening for food material components that enhance cell proliferation, when culturing bovine kidney cells in serum-free medium.

FIG. 8 is a set of photographs showing myosin heavy chain (MyHC) and nuclear (DAPI) fluorescent staining for differentiation-inducing treatment of myoblasts grown in 10% FBS-added medium (10% FBS) and whey-added serum free medium (whey).

FIG. 9 shows cell counts obtained after culturing myoblasts in 10% FBS-added medium and 0.1% whey-added serum free medium, prepared by heat treatment of a FBS stock solution and a 1% whey solution and addition of each to basal medium after heat treatment, and in unheated 10% FBS-added medium and 0.1% whey-added serum free medium without heat treatment.

### MODE(S) FOR CARRYING OUT THE INVENTION

The present invention relates to a cell proliferation medium containing basal medium and whey as a cell proliferation promoting agent. According to a different aspect, the invention relates to a method of preparing cells for producing cultured meat, the method including a step of culturing cells in cell proliferation medium containing basal medium and whey as a cell proliferation promoting agent, and to a method for producing cultured meat from the prepared cells. According to yet another aspect, the invention relates to a cell proliferation promoting agent for producing cultured meat which contains whey. According to yet another aspect, the invention relates to a method of producing a medium for producing cultured meat, wherein the method comprises a step of mixing basal medium with whey as a cell proliferation promoting agent to acquire a cell proliferation medium; and a step of heat sterilizing the medium.

### [Cell Proliferation Medium]

The cell proliferation medium of the invention contains basal medium and whey as a cell proliferation promoting agent. Because whey is a food material, cells cultured in the medium of the invention are highly safe for foods. Moreover, the low cost of whey as a raw material provides the advantage of lower cost for preparation of the medium of the invention. Adding whey can enhance cell proliferation activity. Cells cultured in the medium are highly safe for foods and can therefore be used for producing cultured meat. The medium of the invention is serum-free medium that, while including whey, also lacks animal-derived serum.

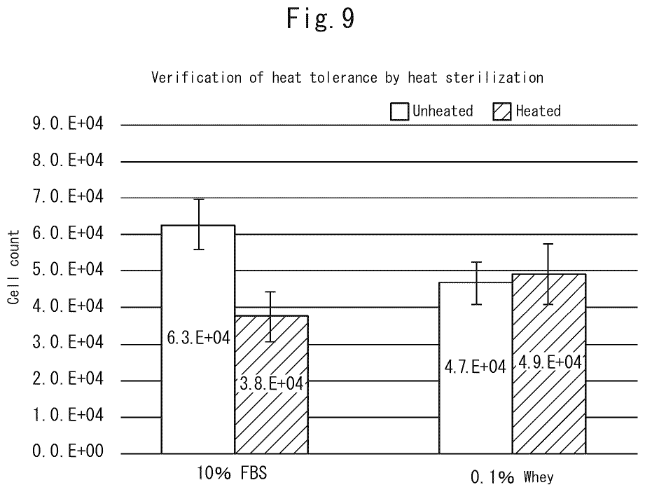

Animal-derived serum is serum produced from animal blood. The supernatant liquid obtained by coagulating harvested blood is referred to as “serum”. Animal-derived serum may be serum derived from any animal such as a cow, horse, goat, donkey, rabbit or bird, but it may particularly refer to bovine serum (BCS) or fetal bovine serum (FBS). Serum comprises proteins such as albumin and globulin, and serum lipids such as triglycerides, cholesterol, phospholipids and free fatty acids, as well as hormones, cytokines and growth factors. Fetal serum contains abundant amounts of components necessary for cell proliferation and is commonly added to medium in the fields of research and medicine. Medium lacking animal-derived serum is referred to as “serum-free medium”. Serum-free medium lacks animal-derived serum but may contain purified components derived from serum, or recombinant proteins from serum-derived components. Animal-derived serum is susceptible to thermal degradation, exhibiting reduced activity after heat sterilization (FIG. 9). Medium containing animal-derived serum is therefore commonly sterilized using filter sterilization or UV sterilization instead of heat sterilization.

According to the invention it is possible to obtain high cell proliferation activity even with serum-free medium, by addition of whey to basal medium. The whey of the invention may therefore be considered to be a cell proliferation promoting agent (also referred to as “cell culture supplement”). The cell proliferation promoting agent of the invention can be used in cell culturing for production of cultured meat and may be added to animal-derived serum-free medium. The cell proliferation promoting effect of whey is not attenuated by boiling (FIG. 9). It is therefore possible to carry out heat sterilization when using whey-containing medium. Heat sterilization of a culture medium can be accomplished using a device such as a plate heat exchanger or steam sterilizer. Convenient sterilization treatment of a medium is desirable for industrial culturing in which mass culturing is a necessity. When heat sterilization is an option, it becomes possible to carry out direct heat sterilization of the prepared medium using a steam sterilizer, or sterilization in the flow channel using a plate type heat exchanger, for introduction into the culturing tank, thus simplifying the steps from medium preparation to culturing. Thermal degradable components may be sterilized by separate filter sterilization or UV sterilization and added to the heat sterilized medium.

Whey is a water-soluble solution prepared by removing the solid components from milk. Whey is inexpensive because it is produced in large quantities as a by-product during the process of producing dairy products such as cheese and yogurt. More specifically, coagulants such as rennet are added to milk or fermented milk to cause solidification and form curd, from which the solid components are separated to obtain whey. The removed solid components are part or all of the milk fat or proteins such as casein. The main components of whey are lactoglobulin, lactalbumin and lactoferrin, but numerous minor components such as free amino acids, inorganic salts and vitamins are also present.

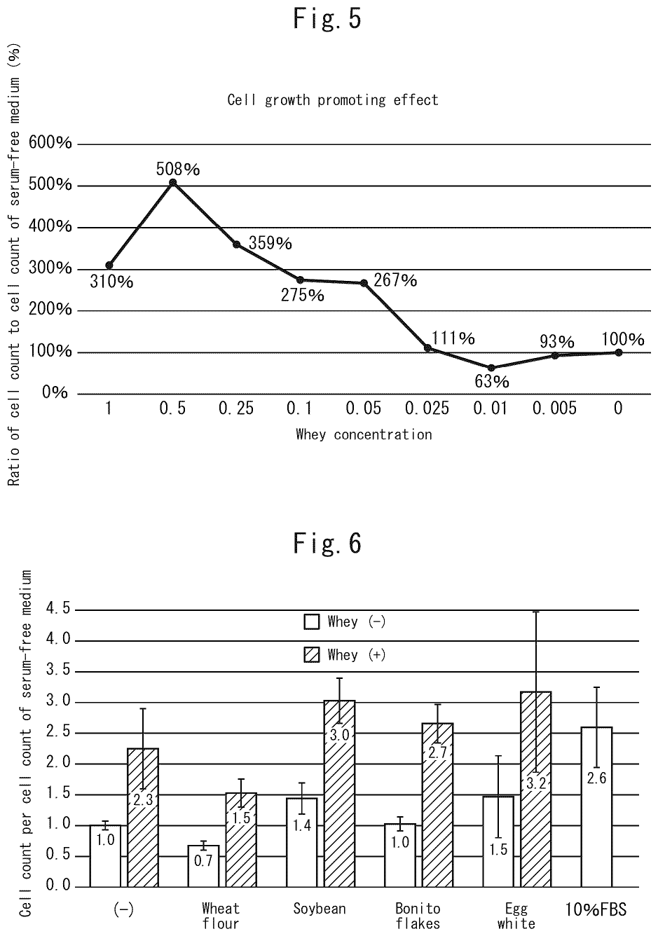

The whey used for the invention may be whey derived from any mammal. Examples are whey products obtained from milk of a cow, horse, goat, sheep, human or donkey. Cow whey is most suitable for use because of its availability. The whey may be liquid or in dry whey powder form. From the viewpoint of addition as a cell proliferation promoting agent, a dry powder form is preferred as it can reduce transport cost. The dry whey powder used may be a commercially available product, or it may be prepared by freeze-drying whey. When dry whey powder is to be used as a proliferation promoting agent, it is added to basal medium at 0.0025 mass % to 1.0 mass %. The concentration of the whey is preferably 0.025 mass % or greater and more preferably 0.05 mass % or greater from the viewpoint of exhibiting a proliferation effect. It is also preferably 0.8 mass % or lower and more preferably 0.5 mass % or lower from the viewpoint of plateau of the proliferation promoting effect. When liquid whey is to be used, the amount added may be determined in terms of the amount of dry powder (FIG. 5).

Basal medium is medium for cell culturing, containing the minimum necessary components for maintenance and growth of the cells. By seeding the cells in basal medium, it is possible to maintain them alive, allowing the cells to proliferate. A number of options are commercially available as basal media, but most generally contain amino acids, vitamins, buffers, inorganic salts and carbon sources. Essential amino acids and non-essential amino acids are both included. Vitamin B1, vitamin C, nicotinic acid and folic acid are included as vitamins. HEPES is included as a buffer. Carbon sources that may be added are monosaccharides such as glucose, disaccharides such as sucrose, oligosaccharides, and polysaccharides. Cell culture medium can generally be prepared by addition of additives such as serum to basal medium. The basal medium used may be any basal medium known in the technical field, examples of which include Dulbecco's Modified Eagle's Medium (DMEM), Basal Medium Eagle (BME), RPMI 1640 medium, DMEM/F12 medium, F10 medium, F12 Ham's Medium, MEM, M199 medium, Ames medium, Iscove's modified medium, Glasgow modified medium and Fischer's medium.

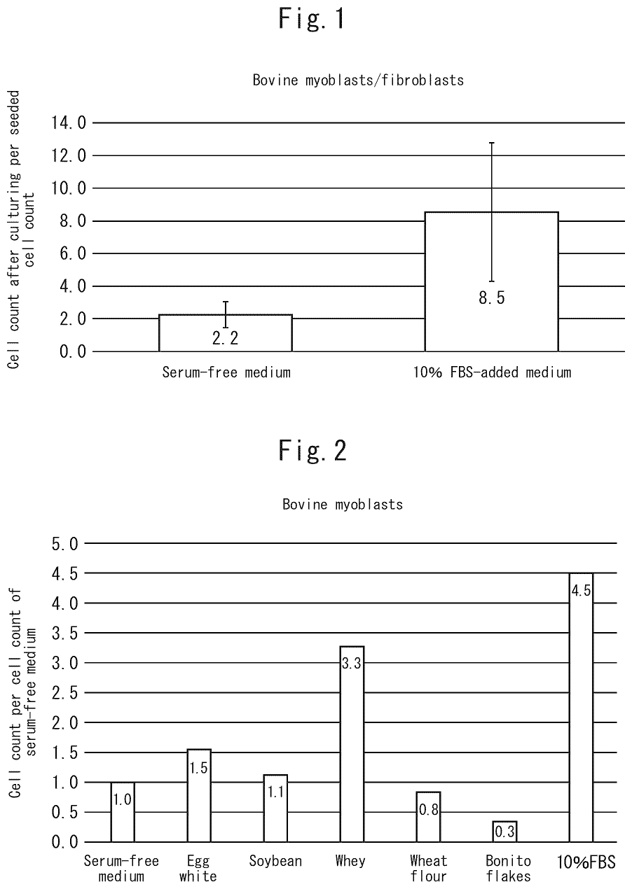

A cell proliferation promoting agent is added to basal medium for cell culturing. For conventional cell culturing, serum such as fetal bovine serum (FBS) is added as a cell proliferation promoting agent (FIG. 1). The cell proliferation medium of the invention, however, contains whey as a cell proliferation promoting agent. An example of the cell proliferation medium of the invention contains no animal-derived serum, and instead contains whey. According to the invention, additives other than whey may also be added to the medium. Such additives include components known to be added to serum-free medium in the technical field. Examples of additives added to serum-free medium in the technical field include lipids, hormone agents, growth factors, cytokines, serum-derived proteins and antibiotics. Hormone agents include dexamethasone. Growth factors include FGF, IGF and insulin, or any of their respective families. Cytokines include IL-1α and IL-1β, which may be added at a concentration of 0.1 to 1000 ng/ml, for example. Serum-derived proteins include fetuin, fibronectin, albumin and globulin, which may be added at a concentration of 0.0001 to 1%, for example. Antibiotics include penicillin and streptomycin which may be added at a concentration of 10 to 500 U/ml for penicillin or 10 to 500 g/ml for streptomycin, for example. ITS (insulin-transferrin-sodium selenite), an additive commonly used in serum-free media or low-serum media, may also be added to the whey-containing serum-free medium of the invention. It may be added in an amount for a concentration of 0.1 to 5% as a 100-fold concentration premix solution, for example.

When whey is to be added to basal medium as a cell proliferation promoting agent, other food material components may be added as well. Optional components may also be added so long as effects suitable for cell culturing are exhibited. Effects suitable for cell culturing include a differentiation inhibiting effect and a proliferation promoting effect, for example. Preferred examples are components that increase cell proliferation activity compared to addition of whey alone, such as components derived from egg white, soybean, wheat flour and fish powder such as bonito flakes. Such food components may be added as extracts, or they may be added as dry powders with filter-removal of the insoluble components. From the viewpoint of exhibiting a high cell proliferation promoting effect, preferred combinations are whey and soybean, whey and bonito flakes, and whey and egg white, with combinations of the whey and egg white or whey and soybean being especially preferred (FIG. 6). Addition of such combinations to basal medium as cell proliferation promoting agents can exhibit a higher cell proliferation promoting effect than 10% fetal bovine serum (FBS). Dry powder of egg white, soybean, wheat flour or bonito flakes may be added to basal medium at 0.0025 mass % to 1.0 mass %. These food material components are preferably added at 0.005 mass % or greater and more preferably 0.01 mass % or greater from the viewpoint of exhibiting a proliferation effect. From the viewpoint of avoiding aggregation of the components, the amount is also preferably 0.5 mass % or lower and more preferably 0.1 mass % or lower. The mass ratio of whey to the other food material components may be appropriately selected in a range of 10:1 to 1:10. The range is preferably 5:1 to 1:5 and more preferably 3:1 to 1:3.

### [Cells]

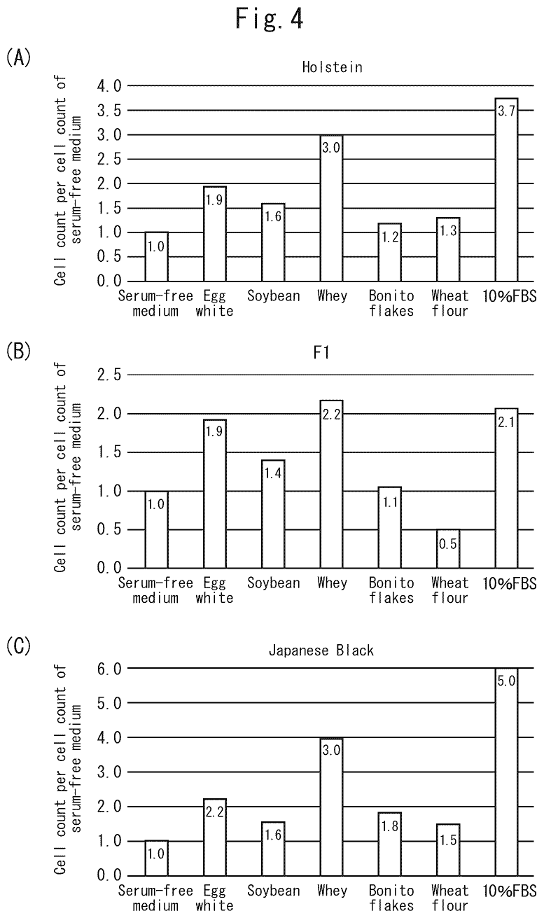

The cell culture medium of the invention allows culturing of any type of animal cells. From the viewpoint of cultured meat production, cells derived from livestock such as cows, pigs, goats, sheep, rabbits, chickens, ostriches or wild ducks may be used. Particularly when using cow cells, the cells may be from any breed such as Holstein, Jersey, Japanese Black, Japanese Brown, Shorthorn or Japanese Polled breeds, as well as hybrids thereof, although cells from the meat breeds of Japanese Black, Japanese Brown, Shorthorn and Japanese Polled breeds are preferred from the viewpoint of production of edible meat. Any cells of these animals may be cultured (FIG. 4). The cell culture medium of the invention also allows culturing of tissues as aggregates of cells. The animal cells may be primary cells harvested from animals, or subcultured cells subcultured from the primary cells, or an established cell line. Primary cells can be harvested by chopping animal tissue in medium. The cells may also be cells differentiated from stem cells such as somatic stem cells, embryonic stem cells or induced pluripotent stem cells. From the viewpoint of cultured meat production, the culturing is preferably of one or more types of cells selected from the group consisting of fibroblasts, adipose tissue-derived cells and muscle tissue-derived cells (FIGS. 2 to 4).

Fibroblasts are cells composing connective tissue which produce components of the extracellular matrix such as collagen and elastin. Muscle fibroblasts are referred to as “myofibroblasts”. Myofibroblasts form connective tissue surrounding muscular fiber bundles in skeletal muscle. Myofibroblasts express α-SMA, and produce the extracellular matrix while also being able to accumulate fat, thus contributing to food chewiness and taste.

Adipose tissue-derived cells are cells composing adipose tissue, and they are cultured after separation from adipose tissue. Adipose tissue-derived cells are of at least one type selected from the group consisting of adipose stem cells, multilocular adipocytes and unilocular adipocytes. Adipose stem cells are mesenchymal stem cells with the ability to differentiate into a variety of different cells, such as muscle cells, adipocytes and connective tissue cells. Multilocular adipocytes, also known as brown adipocytes, contribute to fat combustion in the body. Unilocular adipocytes, also known as white adipocytes, store intracellular fat droplets. Because they contain fat, adipose tissue-derived cells contribute to the desirable taste of meat.

Muscle tissue-derived cells are cells composing muscular tissue, and they are cultured after separation from muscular tissue. Muscle tissue-derived cells include myoblasts, muscle satellite cells and myotube cells, with myoblasts and/or muscle satellite cells being preferred from the viewpoint of proliferation since myotube cells do not have proliferative ability. Muscle satellite cells are intramuscular somatic stem cells which can proliferate and differentiate into myoblasts. Myoblasts are muscular fiber-derived cells, being mononuclear cells with proliferative ability. Upon differentiation of myoblasts, the myoblasts fuse together forming multinucleated myotube cells which further mature into muscular fibers. The structural units of muscular fibers are myofibrils, composed of the muscular proteins of actin fibers and myosin fibers, and depending on the isoform of the myosin they are classified as either red muscle fibers (type I and type IIA) or white muscle fibers (IIB), which also contribute to the taste of meat.

### [Cultured Meat]

Cultured meat is edible meat produced by cell culturing. The phrase “for producing cultured meat” as used herein means that the method is to be used for production of cultured meat and must be acceptable in terms of food hygiene. In terms of food hygiene, it is preferable to avoid the use of animal-derived serum, hormone agents and recombinant proteins. The term “edible meat” generally refers to muscular fibers, connective tissue and fat aggregates. Although it is desirable for cultured meat to mimic the structure of normal meat, it does not necessarily have to include the entire structure of meat, as it is sufficient to include at least one cultured cell type selected from the group consisting of fibroblasts, adipose tissue-derived cells and muscle tissue-derived cells. The cultured product more preferably includes multiple types of cells. Cultured meat may also contain the extracellular matrix in addition to the one or more types of cultured cells selected from the group consisting of fibroblasts, adipose tissue-derived cells and muscle tissue-derived cells. The method for producing cultured meat comprises, for example:
a step of culturing at least one cell type selected from the group consisting of fibroblasts, adipose tissue-derived cells and muscle tissue-derived cells, and
a step of collecting and accumulating the cultured cells. The method for producing cultured meat may also further comprise a differentiation-inducing step and a post-accumulation culturing step. The present invention relates to cultured meat comprising cells cultured in the cell proliferation medium of the invention.

The cell culturing is carried out by seeding cells in the cell proliferation medium of the invention, i.e. medium containing basal medium and whey as a cell proliferation promoting agent. The culturing is conducted under conditions known in the technical field, such as in a CO₂ incubator at 37° C. The culturing may be adherent culture or suspension culture. The cultured cells can be collected as the cultured product by trypsin treatment, and may be further subcultured after collection. Culturing of the cells may be seeding and culturing of the cells on a releasable structure. The structure on which the proliferated cells have attached may also be collected as the cultured product. Such a structure may be formed of extracellular matrix components such as collagen, elastin, fibronectin, laminin and entactin, accumulating a cell-adhering structure to form cultured meat.

The accumulation step includes forming a cultured product of one or more types of collected cells. The cultured product formed in the accumulation step may be a single piece of meat such as steak, or whole meat cuts or minced meat. The accumulation step includes accumulation of the cultured cell product together with one or more substances selected from the group consisting of other cells, blood and tissue. Other cells may be cultured cells or cells harvested from an animal. More specifically, the meat may be formed with a combination of other cells cultured in cell proliferation medium according to the invention. As an example, muscle tissue-derived cells cultured in cell proliferation medium of the invention may be accumulated with adipose tissue-derived cells and/or fibroblasts cultured in cell proliferation medium of the invention. Co-culturing may also be carried out after accumulation. As an example, cultured products of one or more different collected cells may be mixed and then seeded and co-cultured on an extracellular matrix. The extracellular matrix used may be collagen, elastin, fibronectin, laminin and entactin. The medium used in this case may also be cell proliferation medium of the invention.

The accumulation step may be accumulation of cultured products of one or more collected cells with blood and/or tissue. The tissue may be harvested from animals or cultured tissue. As one example, cultured meat may be produced by accumulating blood, adipose tissue or muscular tissue separated during the process of treating meat, together with a cultured product.

The differentiation-inducing step may be carried out after cell culturing, or before the accumulation step, during the accumulation step or after the accumulation step. A differentiation-inducing step allows differentiation of mononuclear muscle satellite cells and myoblasts to multinucleated myotube cells, and further maturation to muscular fibers. Differentiation may be induced by a method known in the technical field, one example being a method of culturing under a high carbon dioxide concentration, such as culturing in a 5 to 10% (v/v) CO₂ atmosphere, to promote differentiation to myotube cells.

### [Method for Producing Medium]

The cell proliferation medium of the invention is prepared by a production method including the following steps:
a step of mixing basal medium with whey as a cell proliferation promoting agent to acquire a cell proliferation medium; and
a step of heat sterilizing the medium.

The production method of the invention may also comprise a step of filter sterilization of the components that are susceptible to heat denaturation, and subsequent addition to the medium. Production of cultured meat requires large-scale culturing of cells, which in turn requires large amounts of medium. Large amounts of medium must be sterilized before cell seeding, and therefore the heat sterilization is preferably by a method that allows convenient, large-scale treatment. Since cell proliferation medium prepared by mixing basal medium with whey is less susceptible to denaturation under heat treatment, it can be provided for heat treatment. The heat treatment may be selected as desired so long as it does not impair the activity of the whey added to the medium, and one example is boiling treatment. The heating temperature may be appropriately selected from the viewpoint of sterilizing the target bacteria, and may be 60° C. to 180° C., for example. It is preferably 75° C. or higher and more preferably 100° C. or higher, from the viewpoint of accomplishing adequate sterilization. From the viewpoint of preventing denaturation of the medium it is also preferably 150° C. or lower and more preferably 130° C. or lower. The heat sterilization time may be selected as appropriate from the viewpoint of accomplishing adequate sterilization. As one example, the heat treatment may be carried out for 0.5 second to 60 minutes. Heat sterilization may also be carried out while medium prepared in a medium preparation tank is being introduced into the culturing tank through a flow channel. A plate type heat exchanger is one example that may be used for heat sterilization in a flow channel.

All of the publications mentioned throughout the present specification are incorporated herein in their entirety by reference. The Examples of the invention described below are intended to serve merely as illustration and do not limit the technical scope of the invention. The technical scope of the invention is limited solely by the description in the Claims. Modifications of the invention, such as additions, deletions or substitutions to the constituent features of the invention, are possible so long as the gist of the invention is maintained.

### EXAMPLES

### Experiment 1: Cell Harvesting

### (1) Harvesting of Myoblasts

Bovine myoblasts were harvested from longissimus muscle by the following steps. Tissue harvested from cows was washed with ethanol and phosphate buffered saline (PBS), and then was cut with scissors on a clean bench for fine chopping. The muscular tissue was digested by incubation with shaking for 1.5 hours at 37° C. in Dulbecco's Modified Eagle's Medium with addition of 0.2% collagenase II (Worthington). The reaction was suspended by addition of 20% FBS to the digested reaction mixture. After centrifugal separation of the digested solution at 80× g for 3 minutes, the floating tissue was removed with forceps, and the supernatant liquid was separated off. The supernatant liquid obtained by further centrifugal separation at 80× g for 3 minutes was passed through a cell sorting nylon mesh (100 μm). The filtrate was centrifuged at 1500×g for 5 minutes to obtain a precipitate, which was suspended in Dulbecco's Modified Eagle's Medium containing 20% FBS. The cell suspension was passed through a 100 μm nylon mesh and then again through a 40 μm nylon mesh, and the filtrate was centrifuged at 1500×g for 5 minutes. The precipitate was allowed to stand for 5 minutes on ice in red blood cell lysate (pluriSelect Life Science), and the blood cells were removed. After washing twice with phosphate buffer, the cells were pooled in Dulbecco's Modified Eagle's Medium containing 10% FBS, and seeded on a culture dish. The proliferating cells were used for the test.

### (2) Harvesting of Adipocytes

Bovine adipocytes were harvested from adipose tissue from near the intestinal tract by the following steps. Tissue harvested from a cow was washed with ethanol and PBS, and then was cut with scissors in a clean bench for fine chopping. The adipose tissue was digested by incubation with shaking for 1 hour in Dulbecco's Modified Eagle's Medium with addition of 0.2% collagenase I (Gibco). After adding 20% FBS to the digested reaction mixture, it was centrifuged at 180×g for 10 minutes. The floating tissue was removed with forceps, and the supernatant liquid was separated off. The supernatant liquid was passed through a cell sorting nylon mesh (100 μm), and then centrifuged at 420×g for 5 minutes. The precipitate was allowed to stand for 5 minutes on ice in red blood cell lysate and the blood cells were removed. After washing twice with phosphate buffer, the cells were pooled in Dulbecco's Modified Eagle's Medium containing 10% FBS, and seeded on a culture dish. The proliferating cells were used for the test.

### (3) Harvesting of Fibroblasts

Fibroblasts were harvested from bovine skin tissue by the following steps. The tissue was washed with ethanol and PBS, and then the dermis layer was peeled off and isolated in a clean bench. The isolated tissue was finely chopped with scissors and allowed to stand on a culture dish containing Dulbecco's Modified Eagle's Medium with addition of 10% FBS, and then cultured for several days in a CO₂ incubator at 37° C. The migrated cells were recovered and used for the test.

### Experiment 2: Comparison of Proliferation Potency in Serum-Free Medium and Serum-Containing Medium

### (i) Medium

The serum-free medium used was Dulbecco's Modified Eagle's Medium with addition of 1% penicillin-streptomycin solution, 1% ITS liquid medium supplement, 2 ng/ml human basic fibroblast growth factor and cell culture lipid additive (Sigma, L0288).

The serum-containing medium used was Dulbecco's Modified Eagle's Medium with addition of penicillin-streptomycin solution and 10% FBS.

### (ii) Proliferation Test

This proliferation test was conducted using primary bovine myoblasts and fibroblasts for a total of 7 different individuals, in both serum-free medium and serum-containing medium. Approximately 5×10³ cells/cm³ were seeded and cultured in a CO₂ incubator set to 37° C., 5% CO₂. After 3 days of culturing, the viable cell count obtained by trypsin treatment was measured and the ratio of cells after proliferation with respect to the number of seeded cells was calculated. The averaged data for the primary bovine myoblasts and fibroblasts from the 7 different individuals is shown in FIG. 1. While the cell proliferation activity of the cells cultured in serum-free medium was low, the cell proliferation activity of the cells cultured in FBS-containing medium was high. The cultured product obtained with the FBS-containing medium had a cell count of about 4-fold compared to the cultured product of the serum-free medium.

### Experiment 3: Search for Food Materials that Enhance Cell Proliferation in Serum-Free Medium

### (1) Search Test Using Bovine Myoblasts

### (i) Medium

Test medium was prepared by adding food components as additives to the serum-free medium prepared in Test 2. The food components used were egg white, soybean, whey, wheat flour and bonito flakes (all in dry powder form). Each food component was dissolved at 0.1% with respect to the serum-free medium (0.02% for the bonito flakes), and the supernatant obtained after centrifugal separation was filtered with a 0.45 μm filter to remove the insoluble components, using the filtrate for the test. As controls there were used additive-free medium and 10% FBS-added medium.

### (ii) Screening Test

Myoblasts derived from Holstein cows were used to search for components that promote proliferation in serum-free medium. Approximately 5×10³ cells/cm³ were seeded and cultured in a CO₂ incubator set to 37° C., 5% CO₂. After 3 days of culturing, the viable cell count obtained by trypsin treatment was measured and the ratio of the cell count in food component-added medium with respect to the cell count in serum-free medium was calculated (FIG. 2). When culturing was in whey-added medium, the cell count was higher than the cultured product in serum-free medium, though lower than the cultured product in 10% FBS-added medium. When culturing was in egg white-added medium, the cell count was higher than the cultured product in serum-free medium, though lower than the cultured product in 10% FBS-added medium.

### (2) Search Test Using Bovine Adipocytes

### (i) Medium

Test medium was prepared by adding food components to the serum-free medium prepared in Test 2. The food components used were egg white, soybean, whey and wheat flour (all in dry powder form). Each food component was dissolved at 0.1% with respect to the serum-free medium, and the supernatant obtained after centrifugal separation was filtered with a 0.45 m filter to remove the insoluble components, using the filtrate for the test. As controls, additive-free (serum-free) medium and 10% FBS-added medium were used.

### (ii) Screening Test

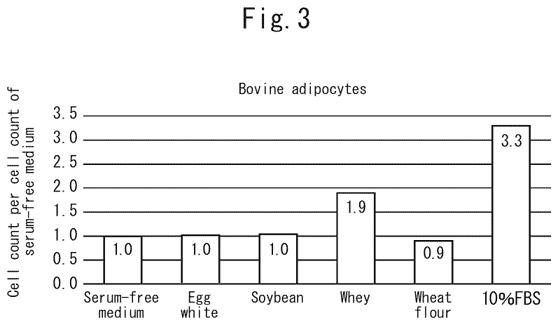

Adipocytes derived from Japanese Black cows were used to search for components that promote proliferation in serum-free medium. Approximately 2×10⁴ cells/cm³ were seeded and cultured in a CO₂ incubator set to 37° C., 5% CO₂. After 4 days of culturing, the viable cell count obtained by trypsin treatment was measured and the ratio of the cell count in food component-added medium with respect to the cell count in serum-free medium was calculated (FIG. 3). When culturing in whey-added medium, the cell count was higher than the cultured product in serum-free medium, though lower than the cultured product in 10% FBS-added medium. The other food components did not affect cell proliferation activity, however.

### (3) Search Test Using Bovine Fibroblasts

### (i) Medium

Test medium was prepared by adding food components as additives to the serum-free medium prepared in Test 2. The food components used were egg white, soybean, whey, wheat flour and bonito flakes (all in dry powder form). Each food component was dissolved at 0.1% with respect to the serum-free medium (0.02% for the bonito flakes), and the supernatant obtained after centrifugal separation was filtered with a 0.45 μm filter to remove the insoluble components, using the filtrate for the test. As controls there were used additive-free (serum-free) medium and 10% FBS-added medium.

### (ii) Screening Test

Fibroblasts from Holstein, Japanese Black and F1 (first-generation hybrid) cows were used to search for components that promote proliferation in serum-free medium. Approximately 5×10³ cells/cm³ were seeded and cultured in a CO₂ incubator set to 37° C., 5% CO₂. After 3 days of culturing, the viable cell count obtained by trypsin treatment was measured and the ratio of the cell count in food component-added medium with respect to the cell count in serum-free medium was calculated (FIG. 4A: Holstein, B: F1, C: Japanese Black). The cell proliferation activity of the food material components varied depending on the cow breed, but still exhibited the same trend. With the Holstein and Japanese Black breeds, the cell count was higher with culturing in whey-added medium compared to the cultured product in serum-free medium, though lower than the cultured product in 10% FBS-added medium. With F1, culturing in whey-added medium was comparable to the cultured product in 10% FBS-added medium. When culturing was in egg white-added medium, the cell count was higher than the cultured product in serum-free medium, though lower than the cultured product in 10% FBS-added medium.

### Experiment 4: Examining Concentration of Whey Added to Serum-Free Medium

### (i) Medium

The serum-free medium used was Dulbecco's Modified Eagle's Medium with addition of penicillin-streptomycin solution, ITS liquid medium supplement, 2 ng/ml human basic fibroblast growth factor, 0.1% cell culture lipid additive and BSA. The food component used was whey (dry powder form). The food component was dissolved in the serum-free medium at different concentrations (1.0 mass %, 0.5 mass %, 0.25 mass %, 0.1 mass %, 0.05 mass %, 0.025 mass %, 0.01 mass %, 0.005 mass % and 0 mass %), and each supernatant obtained after centrifugal separation was filtered with a 0.45 μm filter to remove the insoluble components, and used for the test.

### (ii) Test

Myoblasts from Holstein cows were used to examine the whey concentration that promotes proliferation in serum-free medium. Approximately 5×10³ cells/cm³ were seeded and cultured in a CO₂ incubator set to 37° C., 5% CO₂. After 3 days of culturing, the viable cell count obtained by trypsin treatment was measured and the ratio of the cell count in food component-added medium with respect to the cell count in serum-free medium was calculated (FIG. 5). As a cell proliferation promoting agent, whey exhibited a cell proliferation promoting effect from 0.005 mass %, the cell proliferation promoting effect plateauing at 0.1 mass %.

### Experiment 5: Examining Combined Effect of Whey and Food Components Added to Serum-Free Medium (Bovine Myoblasts)

### (i) Medium

Test medium was prepared by adding food components as additives to the serum-free medium prepared in Test 4. The food components used were egg white, soybean, wheat flour and bonito flakes (all in dry powder form). Each food component was dissolved at 0.1% with respect to the serum-free medium (0.02% for the bonito flakes), and the supernatant obtained after centrifugal separation was filtered with a 0.45 μm filter to remove the insoluble components, using the filtrate for the test. As negative controls there were used food component-free (serum-free) medium and 10% FBS-added medium. The following test was conducted, with further addition of 0.1% whey and without addition of whey to the medium. The whey was added before filtration with a filter.

### (ii) Test

Myoblasts derived from Holstein cows were used to search for components that promote proliferation in serum-free medium. Approximately 5×10³ cells/cm³ were seeded and cultured in a CO₂ incubator set to 37° C., 5% CO₂. After 3 days of culturing, the viable cell count obtained by trypsin treatment was measured and the ratio of the cell count in food component-added medium with respect to the cell count in serum-free medium was calculated (FIG. 6). Whey exhibited a proliferation promoting effect even in combination with other food material components. The proliferation promoting effect was additive, with the combination of soybean and egg white, which have growth promoting effects, exhibiting a growth promoting effect comparable to 10% FBS.

### Comparative Example 1: Search for Food Materials that Enhance Cell Proliferation in Serum-Free Medium (Bovine Kidney Cell Line)

### (i) Medium

Test medium was prepared by adding food components as additives to the serum-free medium prepared in Experiment 2. The food components used were egg white, soybean, whey and wheat flour (all in dry powder form). Each food component was dissolved at 0.1% with respect to the serum-free medium, and the supernatant obtained after centrifugal separation was filtered with a 0.45 μm filter to remove the insoluble components, using the filtrate for the test. As controls there were used additive-free (serum-free) medium and 10% FBS-added medium.

### (ii) Screening Test

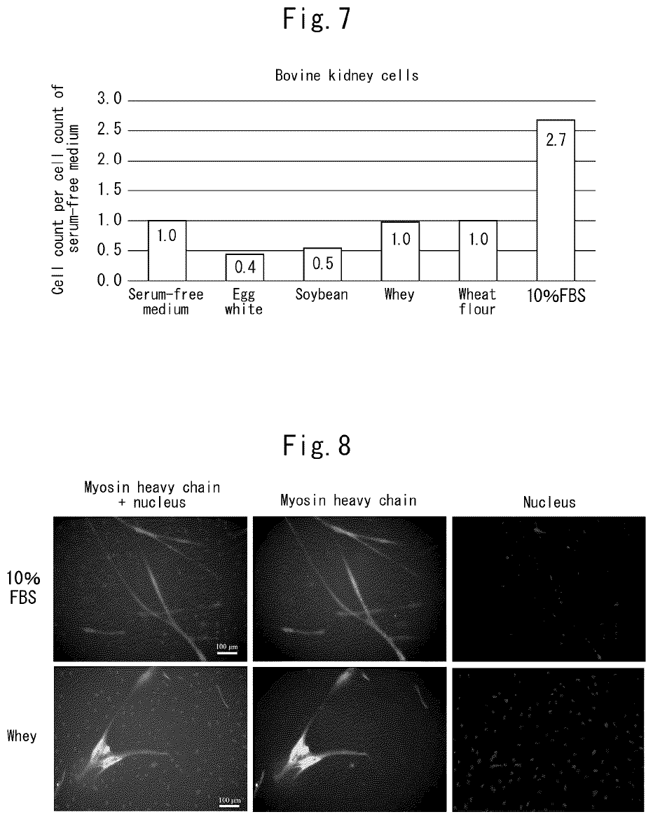

A bovine kidney cell line (MDBK) obtained from ATCC was used to search for components that promote proliferation in serum-free medium. Approximately 1×10⁴ cells/cm³ were seeded and cultured in a CO₂ incubator set to 37° C., 5% CO₂. After 4 days of culturing, the viable cell count obtained by trypsin treatment was measured and the ratio of the cell count in food component-added medium with respect to the cell count in serum-free medium was calculated (FIG. 7). The food material components did not exhibit a proliferation promoting effect for bovine kidney cells.

### Experiment 6: Differentiation of Myoblasts Cultured in Whey Medium

Myoblasts from Holstein cows were used to confirm that cells grown in food component-added medium are induced to differentiate into myotube cells. The food component-added medium used was Dulbecco's Modified Eagle's Medium with addition of penicillin-streptomycin solution, ITS liquid medium supplement, 2 ng/ml human basic fibroblast growth factor, cell culture lipid additive and 0.2% BSA, and with further addition of 0.1% whey powder. The supernatant obtained after centrifugal separation was filtered with a 0.45 μm filter to remove the insoluble components, using the filtrate for the test. The control serum-containing medium used was Dulbecco's Modified Eagle's Medium with addition of penicillin-streptomycin solution and 10% FBS. The differentiation-inducing medium used was Dulbecco's Modified Eagle's Medium with addition of penicillin-streptomycin solution and 2% horse serum. Approximately 0.75×10⁴ cells/cm³ were seeded and cultured for 4 days in a CO₂ incubator set to 37° C., 5% CO₂, and then the medium was exchanged with differentiation-inducing medium and culturing was continued for 6 days. Differentiation to myotubes was confirmed by myosin heavy chain immunostaining.

The immunostaining was carried out by the following steps.
1. The cells were washed once with phosphate buffered saline (PBS) and then incubated overnight at 4° C. in 4% paraformaldehyde for fixing.
2. After washing 3 times with PBS, the cells were treated for 5 minutes with 1% Triton X-100/PBS at room temperature as permeabilizing treatment.
3. After washing 3 times with PBS, commercially available blocking solution for immunostaining (KAC Co., Ltd.) was used for blocking treatment for 30 minutes at room temperature.
4. The cells were then treated for 1 hour at room temperature in a solution containing 1 g/mL of anti-myosin heavy chain monoclonal antibody (Clone MF20), as primary antibody reaction.
5. After washing 3 times with PBS, treatment was carried out with a 500-fold dilution of Alexa 488-labeled goat anti-mouse IgG (Abcam, ab 150117) for 30 minutes at room temperature, as secondary antibody reaction.
6. After washing with PBS and nuclear staining with DAPI, the cells were observed with an All-In-One fluorescent microscope by Keyence Corp. (FIG. 8). It was confirmed that inducing differentiation after proliferation in both whey-added medium and 10% FBS-added medium results in differentiation to multinucleated myotube cells expressing the myosin heavy chain.

### Experiment 7: Heat Tolerance Test for Whey

Heat treated whey and serum were used to prepare medium, and the effect of heat treatment on cell proliferation was evaluated. Used were a 1% whey solution prepared as a solution in water, and inactivated FBS. After submerging the whey solution and FBS dispensed into a 50 ml tube in portions of 10 ml at a time into a boiling pot for 5 minutes and cooling, the solution inside the tube was spined down and used as the heat-treated component. Unheated components (non heat-treated) were used as a control.

The food component-added medium used was Dulbecco's Modified Eagle's Medium with addition of penicillin-streptomycin solution, ITS liquid medium supplement, 5 ng/ml human basic fibroblast growth factor and cell culture lipid additive, and with further addition of a 1/10 amount of whey solution. The supernatant obtained after centrifugal separation was filtered with a 0.45 μm filter to remove the insoluble components, using the filtrate for the test.

The serum-containing medium used was Dulbecco's Modified Eagle's Medium with addition of penicillin-streptomycin solution, and further addition of a 1/10 amount of FBS.

Myoblasts at 5×10³ cells/cm³ were seeded in culture dishes containing different media, and cultured in a CO₂ incubator set to 37° C., 5% CO₂. After 3 days of culturing, the viable cell count obtained by trypsin treatment was measured (FIG. 9). Even when heat-treated, the whey had no change in its cell proliferation promoting effect, whereas FBS has a lower cell growth promoting effect after heat treatment.

1. A cell proliferation medium containing basal medium and whey as a cell proliferation promoting agent.

2. The medium according to claim 1, wherein the proliferated cells are cells to be used for producing cultured meat.

3. The medium according to claim 1, wherein the medium does not contain animal-derived serum.

4. (canceled)

5. The medium according to claim 1, wherein the cells comprise at least one type of cell selected from the group consisting of fibroblasts, adipose tissue-derived cells and muscle tissue-derived cells.

6. The medium according to claim 5, wherein the adipose tissue-derived cells are at least one type of cell selected from the group consisting of adipose stem cells, multilocular adipocytes and unilocular adipocytes.

7. The medium according to claim 5, wherein the muscle tissue-derived cells are at least one type of cell selected from the group consisting of myoblasts and muscle satellite cells.

8. The medium according to claim 1, wherein the cells are bovine cells.

9. The medium according to claim 1, which further comprises a food material component.

10. The medium according to claim 9, wherein the food material component is selected from egg white, soybean, fish powder and wheat flour.

11. A method for preparing cells for producing cultured meat, comprising a step of culturing cells in medium containing basal medium and whey as a cell proliferation promoting agent.

12. The method according to claim 11, wherein the medium does not contain animal-derived serum.

13. (canceled)

14. The method according to claim 11, wherein the medium further comprises a food material component.

15. The method according to claim 14, wherein the food material component-is-selected from egg white, soybean, fish powder and wheat.

16. The method according to claim 11, wherein the cells are bovine cells.

17. The method according to claim 11, wherein the cells comprise at least one type of cell selected from the group consisting of fibroblasts, adipose tissue-derived cells and muscle tissue-derived cells.

18. The method according to claim 17, wherein the adipose tissue-derived cells are at least one type of cell selected from the group consisting of adipose stem cells, multilocular adipocytes and unilocular adipocytes.

19. The method according to claim 17, wherein the muscle tissue-derived cells are at least one type of cell selected from the group consisting of myoblasts and muscle satellite cells.

20. The method according to claim 19, wherein the cells are myoblasts or muscle satellite cells, and wherein the method further comprises a differentiation-inducing step from myoblasts or muscle satellite cells to myotube cells.

21. A method for producing cultured meat, comprising a step of accumulating cells prepared by the method according to claim 11.

22. The method according to claim 21, wherein the prepared cells are accumulated together with one or more substances selected from the group consisting of other cells that are cultured cells or cells acquired from an animal, blood, tissue and extracellular matrix.

23. The method according to claim 22, wherein the other cells are cultured cells or cells acquired from an animal.

24. The method according to claim 21, comprising further culturing after accumulation.

25. A cell proliferation promoting agent for producing cultured meat, comprising whey.

26. The cell proliferation promoting agent according to claim 25, which is added to animal-derived serum-free medium.

27. The cell proliferation promoting agent according to claim 25, which promotes proliferation of at least one type of cell selected from the group consisting of fibroblasts, adipose tissue-derived cells and muscle tissue-derived cells.

28. The cell proliferation promoting agent according to claim 27, wherein the adipose tissue-derived cells are at least one type of cell selected from the group consisting of adipose stem cells, multilocular adipocytes and unilocular adipocytes.

29. The cell proliferation promoting agent according to claim 27, wherein the muscle tissue-derived cells are at least one type of cell selected from the group consisting of myoblasts and muscle satellite cells.

30. The cell proliferation promoting agent according to claim 25, wherein the cells are bovine cells.

31. A method for producing medium, comprising:
a step of mixing basal medium with whey as a cell proliferation promoting agent to acquire a cell proliferation medium; and
a step of heat sterilizing the medium.

32.-33. (canceled)
